(19) United States
(12) Patent Application Publication
(10) Pub. No.: US 2004/0220081 A1
(43) Pub. Date: Nov. 4, 2004

(54) Nanoparticulate bioactive agents

(75) Inventors: Mark R. Kreitz, Providence, RI (US); Yong S. Jong, Providence, RI (US); Edith Mathiowitz, Brookline, MA (US); David J. Enscore, Sudbury, MA (US); Michael J. Bassett, Rumford, RI (US)

Correspondence Address: PATREA L. PABST, PABST PATENT GROUP LLP, 400 COLONY SQUARE, SUITE 1200, ATLANTA, GA, 30361, US

(73) Assignee: Spherics, Inc.

(21) Appl. No.: 10/696,829

(22) Filed: Oct. 30, 2003

Related U.S. Application Data
(60) Provisional application No. 60/423,093, filed on Oct. 30, 2002.
(60) Provisional application No. 60/490,343, filed on Jul. 25, 2003.

(51) Int. Cl. A61K 38/00; A61K 48/00; A61K 31/202; A61K 31/715; A61K 9/14; A61K 31/20

(52) U.S. Cl. 514/2; 514/44; 514/54; 514/558; 264/5; 424/489

(57) ABSTRACT

Bioactive agents may be reproducibly converted into particles having diameters in the range of about 5 to about 2000 nanometers (nm). Conversion is accomplished by dissolving the bioactive agent in a solvent for the bioactive agent, and rapidly altering the polarity of the solution to make it a non-solvent for the bioactive agent, for example by diluting the bioactive agent solution with an excess of a liquid that is a non-solvent for the bioactive agent but is miscible with the solvent. Precipitated bioactive agent nanoparticles are collected by centrifugation, filtration or lyophilization. The nanoparticles have a relatively narrow size distribution, and the average diameter can be controlled by choice of solvent and non-solvent. The nanoparticles are typically amorphous. A surfactant may be added to ensure dispersion of the particles when administered. In the preferred embodiment, the bioactive agent is a drug with low aqueous solubility.

This application claims priority to U.S. Ser. No. 60/423,093 filed Oct. 30, 2002, and U.S. Ser. No. 60/490,343 filed Jul. 25, 2003.

The present invention is directed at compositions containing biologically active agents in the form of nanoparticles, which have enhanced rates of dissolution and/or uptake.

### BACKGROUND OF THE INVENTION

Paclitaxel is a drug of extremely low water solubility and one which exhibits very poor GI absorption upon oral administration. Traditional approaches to parenteral delivery of poorly soluble drugs include using large volumes of aqueous diluents, solubilizing agents, detergents, non-aqueous solvents, or non-physiological pH solutions. These formulations, however, can increase the systemic toxicity of the drug composition or damage body tissues at the site of administration.

For example, paclitaxel is a natural product which has been shown to possess cytotoxic and antitumor activity. While having an unambiguous reputation of tremendous therapeutic potential, paclitaxel has some patient-related drawbacks as a therapeutic agent. These partly stem from its extremely low solubility in water, which makes it difficult to provide in suitable dosage form. Because of paclitaxel's poor aqueous solubility, the current approved (U.S. FDA) clinical formulation consists of a 6 mg/ml solution of paclitaxel in 50% polyoxyethylated castor oil (CREMOPHOR EL™) and 50% dehydrated alcohol. Am. J. Hosp. Pharm., 48:1520-24 (1991). In some instances, severe reactions, including hypersensitivity, occur in conjunction with the CREMOPHOR™ administered in conjunction with paclitaxel to compensate for its low water solubility. As a result of the incidence of hypersensitivity reactions to the commercial paclitaxel formulations and the potential for paclitaxel precipitation in the blood, the formulation must be infused over several hours. In addition, patients must be pretreated with steroids and antihistamines prior to the infusion.

Other efforts directed at enhancing the rate of dissolution have focused on delivering the drug as a dispersion in a water-soluble or biodegradable matrix, typically in the form of polymeric microparticles. For example, the dissolution rate of dexamethasone reportedly was improved by entrapping the drug in chitosan microspheres made by spray-drying (Genta, et al., S.T.P. Pharma Sciences 5(3):202-07 (1995)). Similarly, others have reported enhanced dissolution rates by mixing a poorly soluble drug powder with a water-soluble gelatin, which purportedly makes the surface of the drug hydrophilic (Imai, et al., J. Pharm. Pharmacol., 42:615-19 (1990)). Related efforts have been directed to forming relatively large, porous matrices of low solubility drugs. For example, Roland & Paeratakul, “Spherical Agglomerates of Water-Insoluble Drugs,” J. Pharma. Sci., 78(11):964-67 (1989) discloses preparing beads having a low solubility drug content up to 98%, wherein the beads have a porous internal structure.

Formulations which allegedly have improved delivery characteristics, especially of taxol, including oral delivery, include WO 01/30319, WO 01/57013, WO 01/30448, U.S. Pat. No. 6,334,445, U.S. Pat. No. 6,245,805, WO 98/53811, WO 97/15269, WO 00/78247, and U.S. Pat. No. 5,9698,972. These all remain limited in terms of actual bioavailability. A bioavailability of at least 10% for a taxane administered orally is considered essential for commercial success, although formulations providing lower levels of oral availability may still have applications, especially for preventative therapy of individuals who are at risk of, or who have been treated for, cancer.

One of the factors affecting the dosage of a drug is its rate of dissolution in the body's fluids. Control of the dissolution rate may be important in achieving the desired therapeutic effect. If a drug is readily soluble, its rate of dissolution can be reduced by a variety of controlled release methods. In general, dissolution control methods rely on coating particles of drug with dissolution-retarding coatings or creating tablets that are slow to dissolve or disintegrate. Coatings or tableting materials (e.g. polymers) may be slow to dissolve, or they may be effectively insoluble, and release drug either by in situ degradation of the coating or polymer, or by diffusion of the drug through the coating or polymer.

Control of dissolution rate is a greater challenge when a drug is poorly soluble in bodily fluids. The lack of ready solubility can mean that the bioavailability of the drug is low, especially in oral dosage forms where there is a limited transit time through the gastrointestinal (GI) tract. The goal with such drugs is to find ways to deliver them to the tissue more rapidly than their inherent insolubility allows. One method is to dissolve them in a non-aqueous solvent. Alcoholic extracts or solutions of drugs may be formed. More recently, hydrophobic drugs, such as paclitaxel, have been dissolved in castor bean lipids, and the solution has been emulsified and then injected intravenously as an emulsion. For example, U.S. Pat. No. 6,334,445 to Mettinger describes such a procedure. This procedure has side effects, including allergic reactions to the paclitaxel, reversible myelosuppression, myalgia, mucositis, and alopecia. Therefore, an improvement is needed.

U.S. Pat. No. 6,143,211 to Mathiowitz et al: and U.S. Pat. No. 6,368,586 to Jacob et al. disclose how coated particles of drugs can be used to deliver drugs to the circulation via the intestine. In addition to slow release in the intestines, part of the improvement in delivery is believed to be due to particles that are induced to pass between or through cells of mucosal surfaces (see Mathiowitz et al., Nature 386: 410 (1997)). In addition to the protective effects of the polymeric coating, it appears that smaller particle sizes are taken up more effectively by this process.

However, current methods for preparing drugs as small particles typically produce relatively large particles with diameters in the range of tens of microns up through millimeters. Typically, a drug is produced by precipitation, crystallization, or lyophilization or other forms of drying. The resulting product is usually macroscopic. In standard tableting, the size range of the drug is often not critical. Drugs may be ground, milled, or otherwise comminuted to obtain a reasonably uniform powder for further processing, but particles in the millimeter range are often sufficiently small.

More recently, efforts have been made to produce particles in the submillimeter range, typically for use in controlled release preparations. These methods most commonly involve grinding or milling, although other techniques are known.

U.S. Pat. No. 6,235,224 to Mathiowitz et al. and Mathiowitz et al., Nature 386: 410 (1997) describe a method of encapsulating drugs in micron and sub-micron polymeric microspheres. In this method, called Phase Inversion Nanoencapsulation (“PIN”), a polymer is dissolved in a solvent and the drug or other material to be encapsulated is dissolved or suspended in the polymer solution. The resulting solution or suspension is rapidly diluted with a solution that is a non-solvent for the polymer, and preferably for the drug or agent. The non-solvent is selected to be sufficiently miscible with the solvent so that a single-phase solution that is a non-solvent for the polymer is formed after the dilution. The spontaneous mixing of the two solutions occurs rapidly and with a small characteristic scale of mixing. As a result, the polymer precipitates to form particles with a very small diameter, typically in the range of tens to hundreds of nanometers, or in some cases up to several microns in diameter. These particles are generally uniform in size. The drug or agent is encapsulated in the nanospheres. Upon administration to a patient, or other application, the drug or agent is released from the nanospheres by diffusion, degradation of the polymer, or a combination of these effects.

However in some situations, the presence of an encapsulating polymer may be unnecessary, or even inhibiting, in the delivery of a drug. For example, for the delivery of a highly hydrophobic or otherwise poorly soluble drug, in which the dissolution of the drug is rate-limiting in delivery, no coating is needed to delay drug delivery or protect the drug from the action of a delivery route in the body. Examples of such delivery routes include the circulation system, gastrointestinal tract, urinary and reproductive tracts, mucosa, and skin.

Therefore it is an object of the invention to provide compositions for quick delivery of an agent.

It is a further object of the invention to provide methods of forming particles of an agent to increase the agent's rate of delivery and availability.

It is another object of the present invention to provide a formulation that can-be administered orally to provide clinically acceptable levels of taxanes.

### BRIEF SUMMARY OF THE INVENTION

Biologically active agents may be reproducibly converted into particles having diameters in the range of about 5 to about 2000 nanometers (nm). Conversion is accomplished by dissolving the agent in a solvent for the agent, and rapidly altering the polarity of the solution to make it a non-solvent for the agent particle, for example by diluting the agent solution with an excess of a liquid that is a non-solvent for the agent but is miscible with the solvent. Precipitated agent nanoparticles are collected by centrifugation, filtration or lyophilization. The nanoparticles have a relatively narrow size distribution, and the average diameter can be controlled by choice of solvent and non-solvent. The nanoparticles are typically amorphous. A surfactant may be added to ensure dispersion of the particles when administered. In the preferred embodiment, the agent is a drug. In the most preferred embodiment, the agent is a drug with low aqueous solubility.

### BRIEF DESCRIPTION OF THE DRAWINGS

FIG. 1 is a graph of the particle diameter (μm) versus percent (number and volume) of paclitaxel particles formed by the method disclosed herein.

FIG. 2 is a bar graph comparing the relative bioavailability of paclitaxel (±SEM) for five different oral formulations.

FIG. 3 is a graph of the average relative bioavailability of paclitaxel phase inversion nanoencapsulation (PIN) formulations and stock paclitaxel.

FIG. 4 is a graph comparing the plasma levels of paclitaxel over time following oral administration of paclitaxel-poly(FA:SA) PIN formulations (A), (B), (C) with free paclitaxel.

FIGS. 5a-5d are graphs showing the effect of time following sonication on particle size (FIG. 5a); the effect of sonication time on particle size (FIG. 5b); the effective of drug solution concentration on particle size (FIG. 5c); and the effective solvent:non-solvent ratio on particle size (FIG. 5d).

FIG. 6 is a graph of tumor size following administration of paclitaxel nanoparticles in three different dosages, compared to injection of taxol in cremaphor.

### DETAILED DESCRIPTION OF THE INVENTION

### I. Nanoparticle Compositions

The particles are a population of nanoparticles in which the majority of the population is below one micron in diameter, typically more than 95%, more preferably more than 99%, generally stable, and not aggregating irreversibly.

The particles as prepared have a volume-average diameter less than about 1 micron. These are typically smaller than about 5 microns in diameter (either number average or volume average diameter), more typically in the range of less than 1 micron, and often in the range of about 500 nm to about 50 nm, although smaller average diameters are obtainable. The particle dispersion is relatively narrow, without normally being monodisperse.

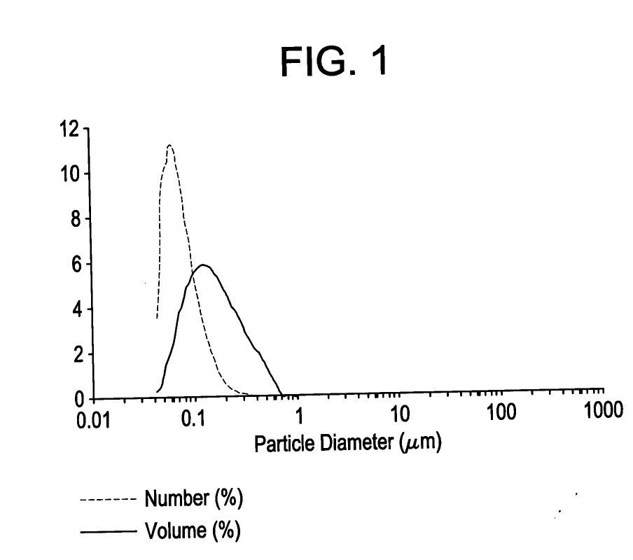

FIG. 1 shows the results of particle sizing on a typical preparation of drug nanoparticles. The number-average diameter is reasonably symmetrical and centered at about 80 nanometers. The volume-average diameter peaks at about 200 nm, and there are effectively no particles larger than about 700 nm.

The nanoparticulates are generally amorphous in structure, in contrast to traditional drug particles, which are typically crystalline. The lack of crystallinity can be observed by microscopy, or by methods such as differential scanning calorimetry (DSC). When the drug in question is poorly soluble in bodily fluids, it is believed that particles which lack of crystallinity have improved drug dissolution rate when compared to crystalline particles of the same drug.

The nanoparticles typically consist essentially of a drug. As generally used herein, “drug” refers to any biologically active agent, including but not limited to classical small-molecule drugs, therapeutically effective proteins, lipids, polysaccharides, proteoglycans, and polynucleotides. The drug may be a therapeutic, a prophylactic, or a diagnostic agent.

Any drug that is capable of being transformed into a microparticulate or nanoparticulate material may be formed into nanoparticles using the method described herein. The drugs may be in either small molecule or macromolecular forms. The drugs may be of low solubility in bodily fluids or soluble in bodily fluids. For example, the extremely small particle size can be useful in delivery as an aerosol to the nasal passages and sinuses, or to the lung. The method is also useful in the preparation of dosage forms of shear-sensitive drugs, such as proteins and nucleic acids. A large number of drugs are known, and are listed in standard compendia such as the Merck Index and the Physicians Desk Reference.

In the most preferred embodiment, the drugs are poorly soluble in bodily fluids. For example, the drug may be soluble in water to less than about 0.1% w/v at room temperature. Their poor solubility may be due to a slow dissolution rate or an inherently poor solubility. These drugs are often hydrophobic, such as drugs in bioavailability classes II and IV. A number of therapeutically important drugs that have poor solubility are known. Examples include taxanes, such as paclitaxel and docetaxel; camptothecin; cyclosporins and related immune response inhibitors; griseofulvin, itraconazole, and related anti-fungal agents; metromidazole and related anti-dysentery agents; dicumarol and related anticoagulants; and steroids, such as androsterone and estradiol. Drugs with low water solubility, such as those with a water solubility similar to the drugs named above, can also be used.

Taxanes are anticancer cytotoxics that stabilize cellular microtubules. Taxane compounds useful in the compositions and methods described herein include paclitaxel and docetaxel, as well as natural and synthetic analogs thereof which possess anticancer or anti-angiogenic activity. Paclitaxel and docetaxel have substantial activity, and one or both of these agents are widely accepted as components of therapy for advanced breast, lung, and ovarian carcinomas.

Formulations may contain taxane in a drug loading of up to 70% by weight. In a preferred embodiment the taxane is present in a drug loading of between 30 and 70% by weight. The taxane may be present in a drug loading of between approximately 30 and 50% by weight. The formulations may contain low levels of drug loading, such as approximately 10 and 30% by weight taxane, or between approximately 1 and 10% by weight taxane.

### B. Excipients and Carriers

The nanoparticles may be used alone, or may be coated with one or more surface-active agents (“surfactants”), polymers, adhesion promoters, or other additives or excipients. They also may be incorporated into tablets or capsules or other dosage forms, or encapsulated. Many different excipients are commonly used in drug formulations. Classes of excipients include, but are not limited to, tableting aids, disintegrants, glidants, antioxidants and other preservatives, enteric coatings, taste masking agents, and the like. References describing such materials are readily available to and well-known by the practitioners in the art of drug formulations. The excipients may be added during any of the steps described below for including surfactants in the particles. For example, the excipient may be added during the formation of the microparticle; during the dispensing of the microparticles to form a dosage form; or during the administration of the microparticles.

The selection of the additives or excipients is determined in part by the projected route of administration. Any of the conventional routes (e.g. inhalation, oral, rectal, vaginal, topical, and parenteral) are suitable for, and may be enhanced by, the use of the nanoparticulate drug formulations. Suitable formulations include oral formulations, aerosols, topical formulations, parenteral formulations, and implantable compositions. In particular, the nanoparticulate dug formulations are particularly suitable for delivering hydrophobic and other poorly-soluble drugs, such as those in bioavailability classes II and IV, by oral or aerosol administration, thereby replacing a parenteral route of administration.

Surfactants

Optionally the nanoparticles contain a surfactant to eliminate or reduce aggregation of the particles. The surfactant adheres to the surface of the nanoparticles. Typically, a surfactant facilitates the dispersion of the nanoparticles in any or all of the initial non-solvent mixtures in which the particle is formed, the medium in which the nanoparticles are taken up for administration, and the medium (e.g. gastrointestinal fluid) into which the particle is later delivered.

Any surfactant may be useful in the nanoparticles. Suitable surfactants include both small molecule surfactants, often called detergents, and macromolecules (i.e. polymers). The surfactant may also contain a mixture of surfactants. In formulations for parenteral administration, the surfactant is preferably one that is approved by the FDA for pharmaceutical uses. In formulations for non-parenteral administration, the surfactant may be one that is approved by the FDA for use in foods or cosmetics.

The surfactant may be present in any suitable amount. In preferred embodiments, effective surfactants are present as only a small weight fraction of the nanoparticles, such as from 0.1% to 10% (wt of surfactant/weight of the drug). However, larger proportions of surfactant may be needed or convenient, thus the surfactant may be present in a weight percent of 20%, 50% or up to about 100% of the weight of the drug, particularly when the particles are small and the total surface area is accordingly large.

Surfactant selection will necessarily be somewhat empirical, and some surfactants may prove to be ineffective in a particular application. For example the examples below demonstrate that 0.5% of sodium lauryl sulfate (SLS), an effective surfactant in many applications, is not effective at dispersing paclitaxel microparticles during passage through the gastrointestinal tract.

Polymers

Suitable polymers include soluble and water-insoluble, and biodegradable and nonbiodegradable polymers, including hydrogels, thermoplastics, and homopolymers, copolymers and blends of natural and synthetic polymers. Representative polymers which can be used include hydrophilic polymers, such as those containing carboxylic groups, including polyacrylic acid. Bioerodible polymers including polyanhydrides, poly(hydroxy acids) and polyesters, as well as blends and copolymers thereof, also can be used. Representative bioerodible poly(hydroxy acids) and copolymers thereof which can be used include poly(lactic acid), poly(glycolic acid), poly(hydroxy-butyric acid), poly(hydroxyvaleric acid), poly(caprolactone), poly(lactide-co-caprolactone), and poly(lactide-co-glycolide). Polymers containing labile bonds, such as polyanhydrides and polyorthoesters, can be used optionally in a modified form with reduced hydrolytic reactivity. Positively charged hydrogels, such as chitosan, and thermoplastic polymers, such as polystyrene also can be used.

Representative natural polymers which also can be used include proteins, such as zein, modified zein, casein, gelatin, gluten, serum albumin, or collagen, and polysaccharides such as dextrans, polyhyaluronic acid and alginic acid. Representative synthetic polymers include polyphosphazenes, polyamides, polycarbonates, polyacrylamides, polysiloxanes, polyurethanes and copolymers thereof. Celluloses also can be used. As defined herein the term “celluloses” includes naturally occurring and synthetic celluloses, such as alkyl celluloses, cellulose ethers, cellulose esters, hydroxyalkyl celluloses and nitrocelluloses. Exemplary celluloses include ethyl cellulose, methyl cellulose, carboxymethyl cellulose, hydroxymethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, cellulose triacetate and cellulose sulfate sodium salt.

Polymers of acrylic and methacrylic acids or esters and copolymers thereof can be used. Representative polymers which can be used include poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

Other polymers which can be used include polyalkylenes such as polyethylene and polypropylene; polyarylalkylenes such as polystyrene; poly(alkylene glycols), such as poly(ethylene glycol); poly(alkylene oxides), such as poly(ethylene oxide); and poly(alkylene terephthalates), such as poly(ethylene terephthalate). Additionally, polyvinyl polymers can be used, which, as defined herein includes polyvinyl alcohols, polyvinyl ethers, polyvinyl esters and polyvinyl halides. Exemplary polyvinyl polymers include poly(vinyl acetate), polyvinyl phenol and polyvinylpyrrolidone.

Polymers which alter viscosity as a function of temperature or shear or other physical forces also may be used. Poly(oxyalkylene) polymers and copolymers such as poly(ethylene oxide)-poly(propylene oxide) (PEO-PPO) or poly(ethylene oxide)-poly(butylene oxide) (PEO-PBO) copolymers, and copolymers and blends of these polymers with polymers such as poly(alpha-hydroxy acids), including but not limited to lactic, glycolic and hydroxybutyric acids, polycaprolactones, and polyvalerolactones, can be synthesized or commercially obtained. For example, polyoxyalkylene copolymers are described in U.S. Pat. Nos. 3,829,506; 3,535,307; 3,036,118; 2,979,578; 2,677,700; and 2,675,619.

These polymers can be obtained from sources such as Sigma Chemical Co., St. Louis, Mo.; Polysciences, Warrenton, Pa.; Aldrich, Milwaukee, Wis.; Fluka, Ronkonkoma, N.Y.; and BioRad, Richmond, Calif., or can be synthesized from monomers obtained from these or other suppliers using standard techniques.

Polymers can be selected based on their bioadhesives properties, for example, as described in U.S. Pat. Nos. 6,197,346; 6,217,908; and 6,235,313to Mathiowitz et al.

The polymers that can be used include both synthetic and natural polymers, either non-biodegradable or biodegradable. Representative synthetic polymers include polyethylene glycol (“PEG”), polyvinyl pyrrolidone, polymethacrylates, polylysine, poloxamers, polyvinyl alcohol, polyacrylic acid, polyethylene oxide, and polyethyoxazoline. Representative natural polymers include albumin, alginate, gelatin, acacia, chitosan, cellulose dextran, ficoll, starch, hydroxyethyl cellulose, hydroxypropyl cellulose, hydroxy-propylmethyl cellulose, hyaluronic acid, carboxyethyl cellulose, carboxymethyl cellulose, deacetylated chitosan, dextran sulfate, and derivatives thereof. Preferred hydrophilic polymers include PEG, polyvinyl pyrrolidone, poloxamers, hydroxypropyl cellulose, and hydroxyethyl cellulose. The hydrophilic polymer is selected for use based on a variety of factors, such as the polymer molecular weight, polymer hydrophilicity, and polymer inherent viscosity.

Wetting Agents Representative examples of wetting agents include mannitol, dextrose, maltose, lactose, sucrose, gelatin, casein, lecithin (phosphatides), gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters (e.g., TWEEN™s), polyethylene glycols, polyoxyethylene stearates, colloidal silicon dioxide, phosphates, sodium dodecylsulfate, carboxymethylcellulose calcium, carboxymethylcellulose sodium, methylcellulose, hydroxyethylcellulose, hydroxy propylcellulose, hydroxypropylmethylcellulose phthlate, noncrystalline cellulose, magnesium aluminum silicate, triethanolamine, polyvinyl alcohol, and polyvinylpyrrolidone (PVP).

Tyloxapol (a nonionic liquid polymer of the alkyl aryl polyether alcohol type, also known as superinone or triton) is another useful wetting agent. Most of these wetting agents are known pharmaceutical excipients and are described in detail in the Handbook of Pharmaceutical Excipients, published jointly by the American Pharmaceutical Association and The Pharmaceutical Society of Great Britain (The Pharmaceutical Press, 1986).

Preferred dispersants include polyvinylpyrrolidone, polyethylene glycol, tyloxapol, poloxamers such as PLURONIC™ F68, F127, and F108, which are block copolymers of ethylene oxide and propylene oxide, and polyxamines such as TETRONIC™ 908 (also known as POLOXAMINE™ 908), which is a tetrafunctional block copolymer derived from sequential addition of propylene oxide and ethylene oxide to ethylenediamine (available from BASF), dextran, lecithin, dialkylesters of sodium sulfosuccinic acid such as AEROSOL™ OT, which is a dioctyl ester of sodium sulfosuccinic acid (available from American Cyanimid), DUPONOL™ P, which is a sodium lauryl sulfate (available from DuPont), TRITON™ X-200, which is an alkyl aryl polyether sulfonate (available from Rohm and Haas), TWEEN™ 20 and TWEEN™ 80, which are polyoxyethylene sorbitan fatty acid esters (available from ICI Specialty Chemicals), Carbowax 3550 and 934, which are polyethylene glycols (available from Union Carbide), Crodesta F-110, which is a mixture of sucrose stearate and sucrose distearate, and Crodesta SL-40 (both available from Croda Inc.), and SA90HCO, which is C₁₈H₃₇CH₂(CON(CH₃)CH₂(CHOH)₄CH₂OH)₂.

Wetting agents which have been found to be particularly useful include Tetronic 908, the Tweens, Pluronic F-68 and polyvinylpyrrolidone. Other useful wetting agents include decanoyl-N-methylglucamide; n-decyl-β-D-glucopyranoside; n-decyl-β-D-maltopyranoside; n-dodecyl-β-D-glucopyranoside; n-dodecyl-β-D-maltoside; heptanoyl-N-methylglucamide; n-heptyl-β-D-glucopyranoside; n-heptyl-β-D-thioglucoside; n-hexyl-β-D-glucopyranoside; nonanoyl-N-methylglucarnide; n-noyl-β-D-glucopyranoside; octanoyl-N-methylglucamide; n-octyl-β-D-glucopyranoside; and octyl-β-D-thioglucopyranoside. Another preferred wetting agent is p-isononylphenoxypoly(glycidol), also known as Olin-10 G or Surfactant 10-G (commercially available as 10 G from Olin Chemicals). Two or more wetting agents can be used in combination.

Bioadhesive Excipients

Adhesion promoters are described in U.S. Pat. No. 6,156,348 to Santos et al.; U.S. Pat. No. 6,197,346 to Mathiowitz et al.; U.S. Pat. No. 6,217,908 to Mathiowitz et al., and U.S. Pat. No. 6,235,313 to Mathiowitz et al. In some preferred embodiments, the adhesion promoters contain hydrophobic polymers that are less hydrophobic than the drug, metals, and/or metal oxides.

The bioadhesive properties of a polymer are enhanced by incorporating a metal compound into the polymer to enhance the ability of the polymer to adhere to a tissue surface such as a mucosal membrane. Metal compounds which enhance the bioadhesive properties of a polymer preferably are water-insoluble metal compounds, such as water-insoluble metal oxides and hydroxides, including oxides of calcium, iron, copper and zinc. The metal compounds can be incorporated within a wide range of hydrophilic and hydrophobic polymers including proteins, polysaccharides and synthetic biocompatible polymers. In one embodiment, metal oxides can be incorporated within polymers used to form or coat drug delivery devices, such as microspheres, which contain a drug or diagnostic agent. The metal compounds can be provided in the form of a fine dispersion of particles on the surface of a polymer that coats or forms the devices, which enhances the ability of the devices to bind to mucosal membranes. The polymers, for example in the form of microspheres, have improved ability to adhere to mucosal membranes, and thus can be used to deliver a drug or diagnostic agent via any of a range of mucosal membrane surfaces including those of the gastrointestinal, respiratory, excretory and reproductive tracts.

Metal compounds which can be incorporated into polymers to improve their bioadhesive properties include water-insoluble metal compounds, such as water-insoluble metal oxides and metal hydroxides, which are capable of becoming incorporated into and associated with a polymer to thereby improve the bioadhesiveness of the polymer. As defined herein, a water-insoluble metal compound is defined as a metal compound with little or no solubility in water, for example, less than about 0.0-0.9 mg/ml.

The water-insoluble metal compounds, such as metal oxides, can be incorporated by one of the following mechanisms: (a) physical mixtures which result in entrapment of the metal compound; (b) ionic interaction between metal compound and polymer; (c) surface modification of the polymers which would result in exposed metal compound on the surface; and (d) coating techniques such as fluidized bead, pan coating or any similar methods known to those skilled in the art, which produce a metal compound enriched layer on the surface of the device. Preferred properties defining the metal compound include: (a) substantial insolubility in aqueous environments, such as acidic or basic aqueous environments (such as those present in the gastric lumen); and (b) ionizable surface charge at the pH of the aqueous environment.

The water-insoluble metal compounds can be derived from metals including calcium, iron, copper, zinc, cadmium, zirconium and titanium. For example, a variety of water-insoluble metal oxide powders may be used to improve the bioadhesive properties of polymers such as ferric oxide, zinc oxide, titanium oxide, copper oxide, barium hydroxide, stannic oxide, aluminum oxide, nickel oxide, zirconium oxide and cadmium oxide. The incorporation of water-insoluble metal compounds such as ferric oxide, copper oxide and zinc oxide can tremendously improve adhesion of the polymer to tissue surfaces such as mucosal membranes, for example in the gastrointestinal system.

In one embodiment, the metal compound is provided as a fine particulate dispersion of a water-insoluble metal oxide which is incorporated throughout the polymer or at least on the surface of the polymer which is to be adhered to a tissue surface. For example, in one embodiment, water-insoluble metal oxide particles are incorporated into a polymer defining or coating a microsphere or microcapsule used for drug delivery. In a preferred embodiment, the metal oxide is present as a fine particulate dispersion on the surface of a microparticle. The metal compound also can be incorporated in an inner layer of the polymeric device and exposed only after degradation or else dissolution of a “protective” outer layer. For example, a core particle containing drug and metal may be covered with an enteric coating designed to dissolve when exposed to gastric fluid. The metal compound-enriched core then is exposed and become available for binding to GI mucosa.

The fine metal oxide particles can be produced for example by micronizing a metal oxide to produce particles ranging in size, for example from 10.0-300 nm. The metal oxide particles can be incorporated into the polymer, for example, by dissolving or dispersing the particles into a solution or dispersion of the polymer prior to microcapsule formation, and then can be incorporated into the polymer during microcapsule formation using a procedure for forming microcapsules such as one of those described in detail below. The incorporation of metal oxide particles on the surface of the microsphere advantageously enhances the ability of the of the microsphere to bind to mucosal membranes or other tissue surfaces and improves the drug delivery properties of the microsphere.

Metal compounds which are incorporated into polymers to improve their bioadhesive properties can be metal compounds which are already approved by the FDA as either food or pharmaceutical additives, such as zinc oxide.

The bioadhesiveness of the particles can also be enhanced as described in U.S. Pat. No. 6,156,348, methods and compositions for enhancing the bioadhesive properties of polymers using organic excipients. The oligomer excipients can be blended or incorporated into a wide range of hydrophilic and hydrophobic polymers including proteins, polysaccharides and synthetic biocompatible polymers. Anhydride oligomers may be combined with metal oxide particles to improve bioadhesion even more than with the organic additives alone. The incorporation of oligomer compounds into a wide range of different polymers which are not normally bioadhesive dramatically increases their adherence to tissue surfaces such as mucosal membranes.

As used herein, the term “anhydride oligomer” refers to a diacid or polydiacids linked by anhydride bonds, and having carboxy end groups linked to a monoacid such as acetic acid by anhydride bonds. The anhydride oligomers have a molecular weight less than about 5000, typically between about 100 and 5000 daltons, or are defined as including between one to about 20 diacid units linked by anhydride bonds. The anhydride oligomer compounds have high chemical reactivity.

The oligomers can be formed in a reflux reaction of the diacid with excess acetic anhydride. The excess acetic anhydride is evaporated under vacuum, and the resulting oligomer, which is a mixture of species which include between about one to twenty diacid units linked by anhydride bonds, is purified by recrystallizing, for example from toluene or other organic solvents. The oligomer is collected by filtration, and washed, for example, in ethers the reaction produces anhydride oligomers of mono and poly acids with terminal carboxylic acid groups linked to each other by anhydride linkages.

The anhydride oligomer is hydrolytically labile. As analyzed by gel permeation chromatography, the molecular weight may be, for example, on the order of 200-400 for fumaric anhydride oligomers and 2000-4000 for sebacic acid oligomers. The anhydride bonds can be detected by Fourier transform infrared spectroscopy by the characteristic double peak at 1750 cm⁻¹ and 1820cm , with a corresponding disappearance of the carboxylic acid peak normally at 1700 cm⁻¹.

In one embodiment, the oligomers may be made from diacids described for example in U.S. Pat. No. 4,757,128 to Domb et al., U.S. Pat. No. 4,997,904 to Domb, and U.S. Pat. No. 5,175,235 to Domb et al., the disclosures of which are incorporated herein by reference. For example, monomers such as sebacic acid, bis(p-carboxy-phenoxy)propane, isophathalic acid, fumaric acid, maleic acid, adipic acid or dodecanedioic acid may be used.

Organic dyes, because of their electronic charge and hydrophilicity/hydrophobicity, may alter the bioadhesive properties of a variety of polymers when incorporated into the polymer matrix or bound to the surface of the polymer. A partial listing of dyes that affect bioadhesive properties include, but are not limited to: acid fuchsin, alcian blue, alizarin red s, auramine o, azure a and b, Bismarck brown y, brilliant cresyl blue ald, brilliant green, carmine, cibacron blue 3GA, congo red, cresyl violet acetate, crystal violet, eosin b, eosin y, erythrosin b, fast green fcf, giemsa, hematoylin, indigo carmine, Janus green b, Jenner's stain, malachite green oxalate, methyl blue, methylene blue, methyl green, methyl violet 2b, neutral red, Nile blue a, orange II, orange G, orcein, paraosaniline chloride, phloxine b, pyronin b and y, reactive blue 4 and 72, reactive brown 10, reactive green 5 and 19, reactive red 120, reactive yellow 2,3, 13 and 86, rose bengal, safranin o, Sudan III and IV, Sudan black B and toluidine blue.

Dispersants

Preferred dispersants include hydrophilic polymers and wetting agents. The amount of dispersant in the formulation is less than about 80%, more preferably less than about 75%, by weight of the formulation The most preferred polymer for use as a dispersant is polyvinylpyrroidone.

### II. Methods of Making the Compositions

### A. Forming Nanoparticles

To make nanoparticles of a drug, the drug is dissolved in a suitable solvent. Then the solution is rapidly diluted by addition to a non-solvent liquid. Generally, the resulting nanoparticles are stable and do not aggregate irreversibly. This simplifies recovery of the particles, which is typically done by methods such as centrifugation and filtration. Then the nanoparticles are redispersed in a suitable solvent prior to use.

The process used to create the nanoparticulate agents is easy to scale up, either as a batch process or as a continuous process.

Solvents

Solvents that are suitable have the properties of dissolving the drug to a useful concentration, which should be at least about 0.5% (w/v), preferably at least about 2% (w/v), and more preferably in the range of 5% to 10% (w/v) or greater than 10% (w/v). Typically, the drug will be dissolved at a concentration that is well below its solubility limit. In addition, the solvent has as low a toxicity as feasible, and is readily removable from the formed product by heat or vacuum. The solvent is fully or at least partially miscible with one or more non-solvents.

Non-solvents

The non-solvent is likewise selected for its low toxicity and easy removability. The key requirements for selecting the non-solvent are that the agent is not soluble in the non-solvent, and that the non-solvent is sufficiently miscible with the solvent to form a single solvent phase after mixing. The ratio of solvent to non-solvent is sufficient to make the mixed solution a non-solvent for the drug. To avoid wastage of drug during processing, the solubility of the drug in the mixed solution is preferably low, for example 0. 1% (w/v), or less. Preferably, the solvent and the non-solvent are selected so that the absolute value of the difference in their solubility parameters is less than about 6(cal/cm³)^{1/2}. For example, if the solvent is an alcohol, such as ethanol, the non-solvent could be water or an aqueous solution. If the non-solvent is not water-miscible, for example dichloromethane (methylene chloride), then a suitable non-solvent could be a non-polar solvent, such as an alkane.

Because the solvent and the non-solvent are miscible, agitation is not required to cause the formation of nanoparticles. In small volumes, the solvent and non-solvent can be mixed sufficiently by pouring one into the other. The drug/solvent solution may be poured into a volume of the non-solvent, or the non-solvent may be poured into a drug/solvent solution. In larger volumes, it may be convenient to stir one liquid while adding the other. Alternatively, particularly for mass production, the solvent and non-solvent can be mixed continuously as flowing streams of appropriate proportions. Agitation, if provided, only needs to be sufficient to disrupt the laminar flow of the streams.

Because of their miscibility, the scale on which the spontaneous mixing of the liquids occurs is small. This is in contrast to the mixing of immiscible liquids, in which surface tension tends to cause coalescence of the non-continuous phase, and in which vigorous agitation is therefore required to reduce particle size. Hence, when the two solutions are mixed, the drug precipitates out as very fine particles, generally with diameters of less than 5 microns.

Any pair of solvent and non-solvent in which the liquids are miscible and chemically and physically compatible with the drug may be used. “Chemical compatibility” refers to the absence of a chemical reaction between the solvent and the drug, aside from reversible changes, such as ionization of acid groups in water. “Physical compatibility” refers to the absence of significant denaturation of macromolecular drugs, such as proteins.

### B. Forming Nanoparticles Containing Surfactant or Other Excipients and Drug

One or more surfactants or other excipients, such as bioadhesives, can be added to the drug in a number of ways. A surfactant may be applied at one or more of several steps in the process of producing and dispensing nanoparticles of the invention. First, the surfactant may be present in the initial solution of drug or other nanoparticle-forming material. Second, it may be present in the non-solvent that is mixed with the drug solution to form the nanoparticles.

A third method involves adding a surfactant to a drug solution before precipitation with a non-solvent. This is a preferred method for small-molecule surfactants.

A fourth method involves dissolving a surfactant in a solvent that is the same as the solvent used to dissolve the drug. Then the surfactant solution is mixed with a non-solvent. The non-solvent is preferably the same as the non-solvent that is mixed with the drug solution; if different, the non-solvent for the surfactant must also be a non-solvent for the drug. The drug solution is mixed separately with a non-solvent. Then the two mixtures of solvent and non-solvent are combined, and the nanoparticles of drug are collected. This method is particularly suitable when the surfactant is a macromolecular surfactant or dispersant.

A fifth method involves allowing some aggregation of the particles during particle collection, and then providing an appropriate dispersant when the particles are taken up for use. This method is particularly useful in medical and veterinary applications. As shown in the examples below, the addition of a suitable disaggregating surfactant can markedly increase the bioavailability of a nanoparticulate drug. The mechanism of this increase is believed to be reversal or prevention of particle aggregation before or during ingestion or injection.

### III. Uses for the Compositions

Any medical or veterinary condition that can be treated by a drug may be treated using the nanoparticulate drugs. In the preferred embodiment, the formulation is administered to treat a disease such as cancer, to administer an oral vaccine, or for any other medical or nutritional purpose requiring uptake through the mucosa of the drug or bioactive to be delivered.

Drug nanoparticles may be administered to a patient by a variety of routes. These include, without limitation, oral delivery to the tissues of the oral cavity, the gastrointestinal tract and by absorption to the rest of the body; delivery to the nasal mucosae and to the lungs (pulmonary); delivery to the, skin, or transdermal delivery; delivery to other mucosae and epithelia of the body, including the reproductive and urinary tracts (vaginal, rectal, ureter); parenteral delivery via the circulation; and delivery from locally implanted depots or devices.

### EXAMPLES

As shown in the examples, the nanoparticulate drug formulations may be used to enhance the delivery of poorly-soluble drugs across the tissues of the intestine, thereby allowing hydrophobic drugs to be delivered orally rather than parenterally.

In the following examples, materials were obtained from laboratory supply houses and were of grades suitable for biomedical research. The material/supplier pairs named herein were selected for convenience. Paclitaxel: Hauser Inc. Span 85 and Span 80: Sigma. PVP (polyvinylpyrrolidone), MW 40,000 (listed), and pentane: EM Science. Dichloromethane: Burdick & Jackson. TWEEN® 20: Malinckrodt. PEG (polyethylene glycol), MW 4500(listed), Spectrum Chemicals. EUDRAGIT® 100, MW 135,000: Rohm & Hass. PLGA (poly lactide-co-glycolide), 50:50, sold as RG502 (MW not stated), Boehringer Ingelheim. Fumaric acid and sebacic acid: Aldrich.

The examples demonstrate that a formulation consisting of nanoparticles and/or microparticles of a taxane such as paclitaxel, preferably encapsulated or dispersed in a biodegradable pharmaceutically acceptable polymer such as poly(lactide-co-glycolide) (“PLGA”), most preferably further including bioadhesive enhancing agents such as FeO, Fe₂O₃, Fe₃O₄, and fumaric anhydride oligomers, and most preferably further including a dispersant such as polyvinylpyrrolidone (“PVP”), has been developed. Through encapsulation in phase-inversion particles, which are taken up by the GI epithelial cells following oral administration, paclitaxel is detectable in the blood and plasma by HPLC analysis. Levels of 5-15% bioavailability are typical. This is a nano- and microparticle formulation for delivering paclitaxel and/or other drugs which are poorly water soluble and/or have poor absorption from the gastrointestinal tract, following oral administration.

The formulation can include polymers such as poly-lactic acid (PLA), poly-lactide-co-glycolide (PLGA), and poly(fumaric-co-sebacic anhydride), with FeO/Fe₂O₃, fumaric anhydride oligomers, poly vinyl pyrrolidone, and paclitaxel, or can be combinations of the above components, including formation of nano/microparticles of a taxane alone. In the preferred method of manufacture, all components but the FeO/Fe₂O₃ are dissolved in an organic solvent, such as dichloromethane, acetone, chloroform, ethyl acetate, and passed through a 0.2 μm PTFE filter. The FeO/Fe₂O₃ is then added and the resulting solution/suspension is bath sonicated for 2-5 minutes. Promptly, the solution/suspension is dumped into a pressure vessel containing a non-solvent such as pentane, hexane, heptane, or petroleum ether, present at a volume of 15-100 times the volume of the organic solvent. The solution/suspension self-disperses, or can be agitated if necessary, forming nano/micro droplets of the solution/suspension. Drug-encapsulated nano/micro particles form quickly and spontaneously as the solvent leaves the droplets and enters the non-solvent. The particles are removed by filtration and vacuum dried to remove residual solvent and/or non-solvent.

These taxane formulations have been tested in vivo, and shown to yield oral bioavailabilities between 5 and 15 percent calculated relative to the IV dose that yields the same plasma AUC as observed for a given oral administration.

### II. Formation of Nano- or Micro-particles

In the preferred embodiment, the formulation is in the form of nano- or microparticles, which may be in the form of microcapsules, microspheres, or microparticles. As noted above, a wide variety of polymers can be used to form microspheres, wherein the polymer surface of the microsphere has incorporated therein a metal compound and/or oligomers which enhance bioadhesive properties of the microsphere such as the ability of the microsphere to adhere to mucosal membranes. The metal compounds, such as water-insoluble metal oxides, and/or oligomers which enhance the bioadhesive properties of the polymers preferably are incorporated into the polymer before formation of the microspheres. As used herein, the term “microspheres” includes microparticles and microcapsules having a core of a different material than the outer wall. Generally, the microspheres have a diameter from the nanometer range up to about 5 mm. For all methods, solvent evaporation, hot melt microencapsulation, solvent extraction, spray-drying, and phase-inversion, the particle size can be affected by the stirring rate to achieve particle sizes preferably smaller than 10micrometers, more preferably smaller than 5 micrometers, more preferably smaller than 2 micrometers, and more preferably smaller than 1 micrometer. The microsphere may consist entirely of bioadhesive polymer incorporating a metal compound such as a water-insoluble metal oxide or can have only an outer coating of bioadhesive polymer incorporating the metal compound or other mucoadhesive agent.

In one embodiment, polylactic acid microspheres can be fabricated using methods including solvent evaporation, hot-melt microencapsulation and spray drying. Polyanhydrides made of bis-carboxyphenoxypropane and sebacic acid or poly(fumaric-co-sebacic) can be prepared by hot-melt microencapsulation. Polystyrene microspheres can be prepared by solvent evaporation. Hydrogel microspheres can be prepared by dripping a polymer solution, such as alginate, chitosan, alginate/polyethylenimine (PEI) and carboxymethyl cellulose (CMC), from a reservoir though microdroplet forming device into a stirred ionic bath, as disclosed in PCT WO 93/21906, published Nov. 11, 1993.

In the preferred embodiment, the particles are nanoparticles formed by phase-inversion, as described in more detail below.

Solvent Evaporation

Methods for forming microspheres using solvent evaporation techniques are described in E. Mathiowitz et al., J. Scanning Microscopy, 4:329 (1990); L. R. Beck et al., Fertil. Steril., 31:545 (1979); and S. Benita et al., J. Pharm. Sci., 73:1721 (1984). The polymer is dissolved in a volatile organic solvent, such as dichloromethane. A substance to be incorporated is added to the solution, and the mixture is suspended in an aqueous solution that contains a surface active agent such as poly(vinyl alcohol). The resulting emulsion is stirred until most of the organic solvent evaporated, leaving solid microspheres. Microspheres with different sizes (1-1000 micrometers) and morphologies can be obtained by this method. This method is useful for relatively stable polymers like polyesters and polystyrene. However, labile polymers, such as polyanhydrides, may degrade during the fabrication process due to the presence of water. For these polymers, some of the following methods performed in completely anhydrous organic solvents are more useful.

Hot Melt Microencapsulation

Microspheres can be formed from polymers such as polyesters and polyanhydrides using hot melt microencapsulation methods as described in Mathiowitz et al., Reactive Polymers, 6:275 (1987). In this method, the use of polymers with molecular weights between 3-75,000 daltons is preferred. In this method, the polymer first is melted and then mixed with the solid particles of a substance to be incorporated that have been sieved to less than 50 micrometers or have been micronized to less than 10 micrometers, preferably to less than 5micrometers, preferably to less than 1 micrometer. The mixture is suspended in a non-miscible solvent (like silicon oil), and, with continuous stirring, heated to 5° C. above the melting point of the polymer. Once the emulsion is stabilized, it is cooled until the polymer particles solidify. The resulting microspheres are washed by decantation with petroleum ether to give a free-flowing powder. Microspheres with sizes between one to 1000 micrometers are obtained with this method.

Solvent Extraction

This technique is primarily designed for polyanhydrides and is described, for example, in PCT WO 93/21906, published Nov. 11, 1993. In this method, the substance to be incorporated is dispersed or dissolved in a solution of the selected polymer in a volatile organic solvent like dichloromethane. This mixture is suspended by stirring in an organic oil, such as silicon oil, to form an emulsion. Microspheres that range between 1-300 micrometers can be obtained by this procedure.

Spray-Drying

Methods for forming microspheres using spray drying techniques are described in U.S. Pat. No. 6,262,034 to Mathiowitz et al. In this method, the polymer is dissolved in an organic solvent such as dichloromethane. A known amount of a substance to be incorporated is suspended (insoluble agent) or co-dissolved (soluble agent) in the polymer solution. The solution or the dispersion then is spray-dried. Microspheres ranging between 1-10 micrometers are obtained. This method is useful for preparing microspheres for imaging of the intestinal tract. Using the method, in addition to metal compounds, diagnostic imaging agents such as gases can be incorporated into the microspheres.

Phase Inversion

Phase inversion nanoencapsulation (PIN) is a process involving the spontaneous formation of discreet microparticles. This one-step process does not require emulsification of a solvent phase in the non-solvent phase. Under proper conditions, low viscosity polymer solutions can be forced to phase invert into fragmented spherical polymer particles when added to appropriate nonsolvents. Phase inversion phenomenon has been applied to produce macro and microporous polymer membranes, hollow fibers, and nano and microparticles forming at low polymer concentrations. PIN has been described by Mathiowitz et al. in U.S. Pat. Nos. 6,143,211 and 6,235,224, each of which are incorporated by reference.

During the formation of the PIN product using the prior art PIN method, noticeable aggregation of the primary particles suspended in the non-solvent may occur within 30 seconds of the initial injection of the polymer solution. The reasons for the aggregation may lie in the interaction between the polymer and the non-solvent or in the interactions of the polymer with itself. Interaction with the non-solvent is polymer dependent. An example is the interaction between PLGA-based PIN particles and n-heptane. PIN particles composed of 12K PLGA (50:50 L:G) aggregate within 30 seconds of injection, while similar particles based on a 20:80 FA:SA polymer material demonstrate less aggregation. This aggregation of primary particles is the most likely causal factor for an increased particle size in the final product upon re-suspension. This particle aggregation may affect overall release of agent or the ability of PIN particles to traverse mucosal epithelia.

The methods of the invention preserve the primary particle size and also produce microparticles characterized by a homogeneous size distribution making a more accurate and reproducible delivery system. Typical microencapsulation techniques produce heterogeneous size distributions ranging from 10 μm to mm sizes. Prior art methodologies attempt to control particle size by parameters such as stirring rate, temperature, polymer/suspension bath ratio, etc. Such parameters, however, have not resulted in a significant narrowing of size distribution. The PIN method can produce, for example, nanometer sized particles which are relatively monodisperse in size. The modified PIN method of the invention reduces the particle size even further by reducing particle aggregation. By producing a microparticle that has a well defined and less variable size, the properties of the microparticle such as when used for release of a bioactive agent can be better controlled. Thus, the invention permits improvements in the preparation of sustained release formulations for administration to subjects.

As described in U.S. Pat. No. 6,235,224, microspheres can be formed from polymers using a phase inversion method wherein a polymer is dissolved in a good solvent, fine particles of a substance to be incorporated, such as a drug, are mixed or dissolved in the polymer solution, and the mixture is poured into a strong non-solvent for the polymer, to spontaneously produce, under favorable conditions, polymeric microspheres, wherein the polymer is either coated on the particles or the particles are dispersed in the polymer. The method can be used to produce microparticles in a wide range of sizes, including, for example, about 100 nanometers to about 10 micrometers. Exemplary polymers which can be used include polyvinylphenol and polylactic acid. Substances which can be incorporated include, for example, imaging agents such as fluorescent dyes, or biologically active molecules such as proteins or nucleic acids.

Protein Microencapsulation

Protein microspheres can be formed by phase separation in a non-solvent followed by solvent removal as described in U.S. Pat. No. 5,271,961 to Mathiowitz et al. Proteins which can be used include prolamines such as zein. Additionally, mixtures of proteins or a mixture of proteins and a bioerodable material polymeric material such as a polylactide can be used. In one embodiment, a prolamine solution and a substance to be incorporated are contacted with a second liquid of limited miscibility with the proline solvent, and the mixture is agitated to form a dispersion. The prolamine solvent then is removed to produce stable prolamine microspheres without crosslinking or heat denaturation. Other prolamines which can be used include gliadin, hordein and kafirin. Substances which can be incorporated in the microspheres include, in addition to the metal compound, pharmaceuticals, pesticides, nutrients and imaging agents.

Low Temperature Casting of Microspheres

Methods for very low temperature casting of controlled release microspheres are described in U.S. Pat. No. 5,019,400 to Gombotz et al. In the method, a polymer is dissolved in a solvent together with a dissolved or dispersed substance to be incorporated, and the mixture is atomized into a vessel containing a liquid non-solyent at a temperature below the freezing point of the polymer-substance solution, which freezes the polymer droplets. As the droplets and non-solvent for the polymer are warmed, the solvent in the droplets thaws and is extracted into the non-solvent, resulting in the hardening of the microspheres.

In addition to the metal compound, biological agents such as proteins, short chain peptides, polysaccharides, nucleic acids, lipids, steroids, and organic and inorganic drugs can be incorporated into the microspheres. Polymers which can be used to form the microspheres include but are not limited to poly(lactic acid), poly(lactic-co-glycolic acid), poly(caprolactone), polycarbonates, polyamides and polyanhydrides. The microspheres produced by this method are generally in the range of 5 to 1000 micrometers, preferably between about 30 and 50 micrometers. But, by using the correct nozzle smaller microspheres could be formed.

Double Walled Microcapsules

Methods for preparing multiwall polymer microspheres are described in U.S. Pat. No. 5,985,354 to Mathiowitz et al. In one embodiment, two hydrophilic polymers are dissolved in an aqueous solution. A substance to be incorporated is dispersed or dissolved in the polymer solution, and the mixture is suspended in a continuous phase. The solvent then is slowly evaporated, creating microspheres with an inner core formed by one polymer and an outer layer of the second polymer. The continuous phase can be either an organic oil, a volatile organic solvent, or an aqueous solution containing a third polymer that is not soluble with the first mixture of polymers and which will cause phase separation of the first two polymers as the mixture is stirred.

Multilayer polymeric drug, protein, or cell delivery devices can be prepared from two or more hydrophilic polymers using the method. Any two or more different biodegradable, or non-degradable, water soluble polymers which are not soluble in each other at a particular concentration as dictated by their phase diagrams may be used. The multilayer microcapsules have uniformly dimensioned layers of polymer and can incorporate a range of substances in addition to the metal compound including biologically active agents such as drugs or cells, or diagnostic agents such as dyes.

Microspheres containing a polymeric core made of a first polymer and a uniform coating of a second polymer, and a substance incorporated into at least one of the polymers, can be made as described in U.S. Pat. No. 4,861,627.

Hydrogel Microspheres

Microspheres made of gel-type polymers, such as alginate, are produced through traditional ionic gelation techniques. The polymer first is dissolved in an aqueous solution, mixed with a substance to be incorporated, and then extruded through a microdroplet forming device, which in some instances employs a flow of nitrogen gas to break off the droplet. A slowly stirred ionic hardening bath is positioned below the extruding device to catch the forming microdroplets. The microspheres are left to incubate in the bath for twenty to thirty minutes in order to allow sufficient time for gelation to occur. Microsphere particle size is controlled by using various size extruders or varying either the nitrogen gas or polymer solution flow rates.

Chitosan microspheres can be prepared by dissolving the polymer in acidic solution and crosslinking it with tripolyphosphate. Carboxymethyl cellulose (CMC) microspheres can be prepared by dissolving the polymer in acid solution and precipitating the microsphere with lead ions. Alginate/polyethylene imide (PEI) can be prepared in order to reduce the amount of carboxylic groups on the alginate microcapsule. The advantage of these systems is the ability to further modify their surface properties by the use of different chemistries. In the case of negatively charged polymers (e.g., alginate, CMC), positively charged ligands (e.g., polylysine, polyethyleneimine) of different molecular weights can be ionically attached.

In the preferred embodiment, a nano- and microparticle formulation for delivering paclitaxel and/or other drugs which are poorly water soluble and/or have poor absorption from the gastrointestinal tract, following oral administration, is made using polymers such as poly-lactic acid (PLA), poly-lactide-co-glycolide (PLGA), and poly(fumaric-co-sebacic anhydride) (poly (FA:SA)), with FeO/Fe₂O₃, fumaric anhydride oligomer, poly vinyl pyrrolidone, and paclitaxel, or combinations of the above components, including formation of nano/microparticles of paclitaxel alone. These are made including preferably enhancers such as metal oxides, bioadhesive oligomers, and a dispersant such as PVP. All components but the FeO/Fe₂O₃ are dissolved in an organic solvent such as, but not limited to, dichloromethane, acetone, chloroform, ethyl acetate, and passed through a 0.2 μm PTFE filter. The FeO/Fe₂O₃ is then added and the resulting solution/suspension is bath sonicated for 2-5 minutes. Alternately, the FeO/Fe₂O₃ could be added along with the other components, filtration or no filtration performed, and the suspension sonicated. Promptly, the solution/suspension is dumped into a pressure vessel containing a non-solvent such as, but not limited to, pentane, hexane, heptane, or petroleum ether, present at a volume of 15-100 times the volume of the organic solvent. The solution/suspension self-disperses, or can be agitated if necessary, forming nano/micro droplets of the solution/suspension. Drug-encapsulated nano/micro particles form quickly and spontaneously as the solvent leaves the droplets and enters the non-solvent. The particles are removed by filtration and vacuum dried to remove residual solvent and/or non-solvent.

The method may be performed by combining a polymer, a dispersant and taxane in an effective amount of a solvent to form a continuous mixture, and introducing the mixture into an effective amount of a non-solvent to cause the spontaneous formation of a nanoencapsulated product. This method is a modified form of the PIN method which incorporates the use of a dispersant.

The term “dispersant” encompasses “solvent- soluble dispersants” as well as “solvent-insoluble dispersants”, can include water-soluble and non-water-soluble agents, and may be micronized to achieve a greater final efficiency. As used herein, a “solvent-soluble dispersant” refers to a solvent-soluble agent that is an organic solid at room temperature or is of ampiphilic nature and that prevents the aggregation/coalescence of the PIN product during its formation and collection. These compounds are added to and are soluble in the polymer solution phase. Solvent-soluble dispersants include, but are not limited to, natural and synthetic water-soluble polymers or glidants, such as polyvinylpyrrolidone (PVP), polyethylene glycol (PEG), starch, and lecithin.

PVP is a preferred solvent-soluble dispersant because it is soluble in the polymer solution phase as well as soluble in water, and is thus precipitated when added to the non-solvent phase. PVP (C₆H₉NO)ₙ (also referred to as povidone, polyvidone, poly[1-(2-oxo-1-pyrrolidinyl)ethylene]) is a synthetic polymer with a range of molecular weights spanning 2500 to 3,000,000. PVP has been used with solid dosage forms, where it serves as a non-toxic binder in tablets. PVP is also water soluble and is commonly used as a suspension stabilizer for many microparticle or microencapsulated formulations. It is accepted as an excipient in most oral dosing since the compound is not absorbed across intestinal or mucosal surfaces, rendering it non-toxic upon consumption.

PVP is added directly to the polymer solution prior to spontaneous particle formation. The PVP can be added in concentrations ranging from 0.1 to 50% of the total polymer content. The existing PIN process allows for a 0.1 to 20% (weight per volume) total polymer concentration in the solvent phase. The PVP is not used in the PIN process to modify the size of the primary polymer particle itself. This particle size is determined by the operating parameters of the PIN process. Instead, the PVP additive prevents the aggregation of these primary particles into larger sized aggregates, which would result in an increased effective particle size. PVP may be used in the initial polymer solution to maintain the original primary particle size, preventing the typical distribution of PIN material made up of particles and aggregates. PVP can achieve this by integrating into the polymer particle matrix itself, or by phase-separating and forming a coat around the primary polymer microparticle.

Additional benefits may also be derived from the use of PVP in the formulations using the PIN process. For poorly water-soluble drugs, the PVP coating may have the additional benefit of modifying the release characteristics of the material by enhancing the solubility of the drug. PVP can be added to the PIN process, allowing the PVP/PIN product to be tableted directly or with additional additives into a dosage form. This dosage form can benefit from the binding properties of the PVP itself and/or its action as a suspension enhancer upon reconstitution.

An insoluble dispersant can also be used. The method is performed using PIN, but the insoluble dispersant is added to the non-solvent rather than the polymer solution. As used herein, a “solvent-insoluble dispersant” refers to an insoluble agent that prevents the aggregation/coalescence of the PIN product during its formation and collection. The solvent-insoluble dispersants are organic or inorganic molecules that are <100 micrometers, preferably <50 micrometers, and most preferably <25 micrometers. These dispersants could be micronized to reduce their particle size prior to addition to the solvent or non-solvent. These agents may or may not dissolve upon reconstitution of the PIN product in water as does PVP, but, like PVP, are pharmaceutically acceptable additives. They also function to reduce the aggregation of particle during PIN. The PIN method may be performed using a solvent soluble dispersant or a solvent insoluble dispersant or both.

Dispersant can be added to the formulation using any of several methods. For example, a mixture of solvent, polymer, dispersant, and taxane-containing water solution is frozen, then dried to remove the water, preferably by vacuum. With subsequent drying of the frozen mixture, the dried mixture is then re-dissolved in a solvent prior to addition to the non-solvent. In a preferred embodiment, the mixture of the solvent, the polymer, the dispersant, and the agent is frozen in liquid nitrogen. The dispersant, regardless of solubility, may be micronized by one or more methods to achieve a smaller particle size, thereby increasing the dispersant's efficiency.

In another embodiment, the dispersant is added to the non-solvent and to the solvent prior to introduction of the solvent mixture to the non-solvent. In still another embodiment, the dispersant is added only to the non-solvent prior to introduction of the solvent mixture to the non-solvent. In still other embodiments, the dispersant is added to the solvent and added to the non-solvent after introduction of the solvent mixture into the non-solvent or the inhibitor is added only to the non-solvent after introduction of the solvent mixture to the non-solvent. In some embodiments, the dispersant concentration in the solvent is between 0.01% and 10% (weight per volume) and in the non-solvent is between 0.1% and 20% (weight per volume).

The solvent:non-solvent volume ratio may be important in reducing particle aggregation or coalescence. A working range for the solvent:non-solvent volume ratio is between 1:10 and 1:1,000,000. In one embodiment, the working range for the solvent:non-solvent is 1:10-1:200.

The resulting particles have an average particle size between 10 nanometers and 10 micrometers. In some embodiments, the particles have an average particle size between 10 nanometers and 5 micrometers. In yet other embodiments, the particles have an average particle size between 10 nanometers and 2 micrometers, or between 10 nanometers and 1 micrometer.

### III. Administration of Formulations

The formulations typically are orally administered to a patient in need thereof, based on the condition to be treated or prevented, and the known pharmacokinetics of the taxane. Administration may also be pulmonary, nasal, rectal or vaginal. Drug may be administered one or more times daily as necessary. The drug particles may be administered as a particular formulation, in a capsule, table, or suspension, using materials and techniques known to those skilled in the art.

The present invention will be further understood by reference to the following non-limiting examples.

### Example 1

### Preparation of Bioadhesive Nano- and Microparticulate Taxane Formulations

Paclitaxel (30% w/w), fumaric anhydride oligomers (10% w/w), PVP (2.8% w/w), and PLGA (45.4% w/w) were dissolved in an amount of dichloromethane and passed through a 0.2-micrometer PTFE filter. Fe₃O₄ (11.8% w/w) was then added and the entire mixture was bath sonicated for 2 minutes. This mixture was promptly dispersed into an amount of non-solvent, which resulted in a solvent volume to non-solvent volume ratio of 1:100. The particles resulting from the phase inversion process were pressure-filtered under Nitrogen gas from the solvent/non-solvent, collected and vacuum-dried to remove residual solvent and/or non-solvent.

### Example 2

### Preparation of Bioadhesive Nano- and Microparticulate Taxane Formulations

The formulations were prepared as described in Example 1 except that the drug content was increased to 50% (w/w) and the contents of the other components were decreased proportionally. The average relative bioavailability was determined to be 4.8% (+/−1.6) (SEM).

### Example 3

### Preparation of Bioadhesive Nano- and Microparticulate Taxane Formulations

The formulations were prepared as described in Example 1 except that the percent PVP content was tripled and the contents of the other components, except for the drug content which remained the same, were decreased proportionally. The average relative bioavailability was determined to be 7.5% (+/−1.3) (SEM).

### Example 4

### Preparation of Bioadhesive Nano- and Microparticulate Taxane Formulations

The formulations were prepared as described in Example 1 except that the percent Fumaric Anhydride Oligomers content was doubled and the content of the other components, except for the drug content which remained the same as described in Example 1, was decreased proportionally. The average relative bioavailability was determined to be 5.9% (+/−0.6) (SEM). When the percent of Fumaric Anhydride Oligomers was tripled and the contents of the other components, except for the drug content which remained the same as described in Example 1, were decreased proportionally, the average relative bioavailability was determined to be 7.8% (+/−1.1) (SEM).

### Example 5

### Preparation of Bioadhesive Nano- and Microparticulate Taxane Formulations using Polyanhydride Base Polymers

Paclitaxel (30%, 50%, and 70% w/w) and poly(fumaric-co-sabacic) acid (poly(FA:SA)) (20:80) were dissolved in dichloromethane, passed through a 0.2-micrometer PTFE filter and the mixture was bath sonicated for 2 minutes. This mixture was promptly dispersed into a non-solvent, which resulted in a solvent volume to non-solvent volume ratio of 1:100. The particles resulting from the phase inversion process were pressure-filtered under Nitrogen gas from the solvent/non-solvent, collected and vacuum-dried to remove residual solvent and/or non-solvent.

### Example 6

### Preparation of Nano- and Microparticulate Taxane Formulations

Paclitaxel was dissolved in dichloromethane to yield a 3% (w/v) solution. This solution was passed through a 0.2-micrometer PTFE filter and bath sonicated for 2 minutes. This solution was promptly dispersed into a non-solvent, which resulted in a solvent volume to non-solvent volume ratio of 1:100. The particles resulting from the phase inversion process were pressure-filtered under Nitrogen gas from the solvent/non-solvent, collected and vacuum-dried to remove residual solvent and/or non-solvent.

### Example 7

### Testing of Bioavailability of Bioadhesive Taxane Formulations Administered Orally to Rats

FIG. 1 is a graph comparing the average relative bioavailability of different oral formulations. Six different paclitaxel-containing oral formulations were tested. The columns on FIG. 1 are described below from left to right. The height of each column represents the average relative bioavailability following oral administration of 48 mg paclitaxel/kg rat. Column A represents the results from the administration of 30% paclitaxel/PLGA -PIN formulation with bioadhesive excipients, which is described in Example 1 (160 mg formulation/kg rat). Column B represents results from the administration of a 30% paclitaxel/PLGA-PIN formulation without any bioadhesive excipients (160 mg formulation/kg rat). Column C represents results from the co-administration of a blank PLGA formulation containing bioadhesive excipients with free paclitaxel (160 mg formulation/kg rat). Column D represents results from the administration of free paclitaxel, agitated in 0.5% SLS/PBS to induce dissolution (48 mg formulation/kg rat). Column E represents results from the administration of paclitaxel micronized by PIN, which is described in Example 6 (48 mg formulation/kg rat). Column F represents results from the administration of a paclitaxel/PLGA-PIN formulation with bioadhesive excipients (160 mg formulation/kg rat). All preparations were re-suspended for administration in 0.5% SLS/PBS, except for paclitaxel micronized by the phase-inversion (PIN) process (Formulation E) and Formulation F, which were re-suspended in distilled water (dH₂O). The excipients in Formulations A, C and F were Fumaric anhydride oligomers, Polyvinylpyrrolidone (PVP), and Iron Oxide (FeO, Fe₂O₃ and/or Fe₃O₄).

An amount of each formulation was administered via oral gavage directly to the stomach of the rats to deliver 48 mg paclitaxel/kg rat. Blood samples were drawn at specified time points into heparinized tubes and centrifuged. The plasma was removed and prepared for paclitaxel content HPLC analysis by liquid-liquid extraction with diethyl ether. The ether was evaporated and samples were reconstituted in HPLC mobile phase of Acetonitrile:Water and injected directly into the HPLC. Plasma paclitaxel concentrations were plotted at respective time points.

Since the low dose (<10 mg/kg) IV pharmacokinetics of paclitaxel are not linear, the bioavailability of the orally administered drug is calculated relative to the IV dose that yields the same plasma area under the curve (AUC) as observed for a given oral administration. To accomplish this, IV pharmacokinetic studies were performed at several doses, the resultant AUC's were determined, and an equation describing the dose/AUC relationship was fit to the data. This allows the calculation of the IV dose corresponding to the observed oral AUC. The fractional bioavailability (BA) of the oral dose is the ratio of the oral formulation's corresponding IV dose to the actual oral dose (IV dose/Oral dose).

Formulation A resulted in a 8.5% average relative bioavailability. Formulation B resulted in a 3.8% average relative bioavailability. Formulation C resulted in a 1.0% average relative bioavailability. Formulation D did not result in any (0.0%) average relative bioavailability. Formulation E resulted in a 2.4% average relative bioavailability. Formulation F resulted in a 8.5% average relative bioavailability.

The relative bioavailability of paclitaxel/PLGA with excipients made using PIN was 8.5% (see FIG. 1, columns A and F). This result strongly contrasts the bioavailability of paclitaxel/PLGA without excipients which was 3.8% (column B) or paclitaxel alone at 2.4% (column E). Thus, the presence of PLGA and excipients clearly increases the relative bioavailability of the drug. Further, there appears to be no difference in relative bioavailability if the paclitaxel/PLGA was dispersed in 0.5% SLS/PBS (column A) or dH₂O (column F).

### Example 8

### Testing of Bioavailability of Bioadhesive Taxane Formulations Administered Orally to Rats

Formulations containing paclitaxel and poly(FA:SA) (20:80), which are described in Example 5, were tested in vivo in rats. The formulations were re-suspended for administration in 0.5% SLS/PBS.

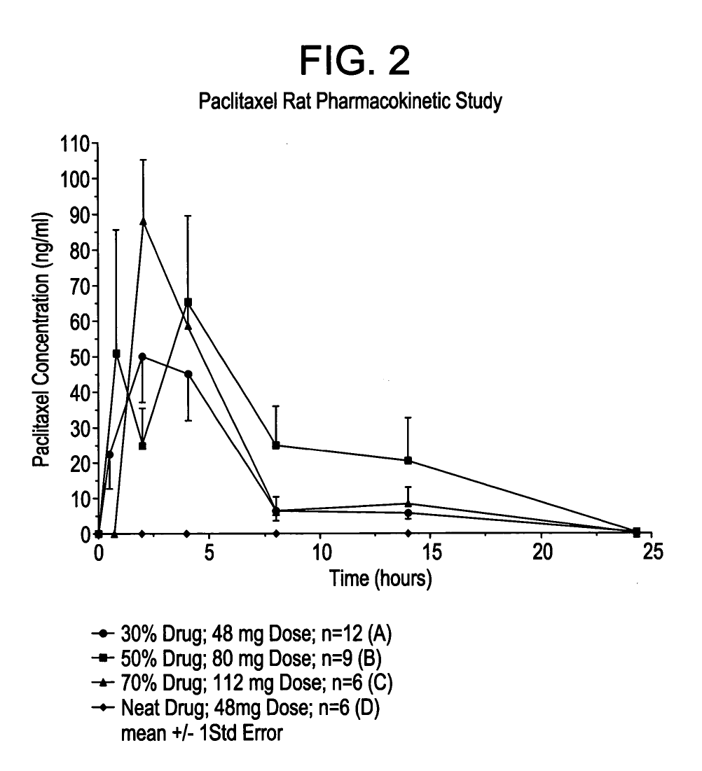

Each re-suspended formulation was then administered via oral gavage directly to the stomach of the rat. Blood samples were drawn at specified time points (1 hour, 2 hours, 4, hours, 8, hours, 14 hours, and 24 hours) into heparinized tubes and centrifuged. The plasma was removed and prepared for paclitaxel content HPLC analysis by liquid-liquid extraction with diethyl ether. The ether was evaporated and samples were reconstituted in HPLC mobile phase of Acetonitrile:Water and injected directly into the HPLC. Plasma paclitaxel concentrations are plotted at respective time points on FIG. 2.

FIG. 2 is a graph comparing the plasma levels of paclitaxel over time following oral administration of paclitaxel-PIN formulations (A), (B), (C), which are described in Example 5, with free paclitaxel (D). Formulation A contained 30% (w/w) paclitaxel/ poly(FA:SA) PIN (48 mg paclitaxel/kg rat). Formulation B contained 50% (w/w) paclitaxel/ poly(FA:SA) PIN (80 mg paclitaxel/kg rat). Formulation C contained 70% (w/w) paclitaxel/ poly(FA:SA) PIN (112 mg paclitaxel/kg rat). Formulation D contained free paclitaxel orally administered at a drug dosage equivalent to Formulation A (48 mg/kg rat). 160mg/kg rat of formulations A, B, and C; and 48 mg/kg rat of formulations D were administered to the rats.

Plasma paclitaxel levels peaked between 0 and 5 hours for all doses tested. As depicted in FIG. 2, the 50% drug concentration (Formulation B) appears to give a higher steady-state plasma concentration between 7 and 20 hours than both the 30% (Formulation A) and 70% (Formulation C) drug concentrations. Paclitaxel was detectable in plasma samples at all concentrations (Formulations A, B, and C) tested up to 24 hours post-administration. In contrast, no paclitaxel was detected in the plasma samples after the administration of free paclitaxel (Formulation D).

### Example 9

### Manufacture and Sizing of Particles

Paclitaxel (3 mg) was dissolved in 1 ml of dichloromethane (methylene chloride) to yield a 0.3% (w/v) solution. This solution was dispersed into 50 ml of petroleum ether, a non-solvent.

The particles resulting from the phase inversion process were recovered from the solvent/non-solvent mixture by pressure-filtration on a 0.2 micron PTFE filter under nitrogen gas, collected from the filter, and vacuum-dried to remove residual solvent and non-solvent. Although many particles were less than 0.2 micron in diameter, the particles formed a filter cake bridging the pores of the membrane, and were recovered in high yield.

### Example 10

### Precipitation of Taxol Particles with Pentane as Non-solvent

Paclitaxel (30 mg) was dissolved in 1 ml of dichloromethane to form a 3% w/v solution. The solution was poured into 100 ml of pentane, the non-solvent. Particles were collected as described in Example 9.

### Example 11

### Precipitation of Taxol Particles with Water as Non-solvent

Paclitaxel (30 mg) was dissolved in 1 ml of acetone and poured into 100 ml of water. Particles were collected by filtration as described in Example 9.

Paclitaxel (30 mg) was dissolved in 1 ml of ethanol and poured into water (100 ml), and the particles were collected by filtration. This demonstrates that the non-solvent may be either more polar than the solvent or less polar, provided that the solvent and non-solvent are substantially miscible.

### Example 12

### Co-precipitation of Taxol with Surfactants

Paclitaxel (90.4 mg), 5.0 mg of polyvinylpyrrolidone (PVP), and 5.1 mg of PLGA (poly lactide-co-glycolide) were dissolved in 3.3 ml of dichloromethane. The solution was pored into 315 ml of pentane, the non-solvent. Particles were collected by filtration and vacuum dried.

### Example 13

### Collection Method Alternatives; Surfactant in Nonsolvent

Paclitaxel (100.4 mg) was dissolved in 3.3 ml of dichloromethane and precipitated in 330 ml of pentane containing 1.65 g of Span 80 (a brand of polyoxyethylated fatty alcohol). Particles were collected by centrifugation, frozen in liquid nitrogen, and vacuum-dried.

### Example 14

### Precipitation of Particles using Other Surfactants

The experiment described in Example 13 was repeated, with 16.7 g of Span 80 in the solvent (and none in the non-solvent), 33.3 mg of EUDRAGIT® S-100 (a polyacrylate), 33.3 mg of PVP, or 33.3 mg of polyethylene glycol (PEG), in each case with similar results.

In another experiment, 300 mg of paclitaxel was dissolved in 6 ml of ethanol and the solution was poured into 30 ml of water. Then 200 mg of TWEEN® 20 was added to the particle suspension. The particles were collected by filtration and vacuum dried.

### Example 15

### Scale up of Formation of Paclitaxel Particles

Paclitaxel (2400 mg) was dissolved in 80 ml of dichloromethane and poured into 8000 ml of pentane. The particles were collected by filtration and vacuum dried.

The values used in these experiments (3%, 1:100) were selected for convenience and maintained for comparability. It appears that the concentration of drug in the solvent, and the amount of non-solvent, can each be significantly reduced.

### Example 16

### Addition of Tissue Adhesive to Particles

A copolymer of fumaric anhydride and sebacic anhydride (pFA:SA) (20:80), prepared in house by conversion of the acids to anhydrides and polymerization of the anhydrides in toluene, essentially according to U.S. Pat. No. 4,891,225, is believed to promote adherence of particles to intestinal and other mucosae. Paclitaxel (2280 mg) and 120 mg of pFA:SA were dissolved in 80 ml of dichloromethane. The solution was poured into 8000 ml of pentane containing 16 ml of Span 85, used as a surfactant. The particles were collected by vacuum and vacuum dried.

### Example 17

### In Vivo Study of Bioavailability of Oral Paclitaxel Formulations

The bioavailabilities of five formulations of orally administered paclitaxel were compared with conventional administration of an intravenous solution containing paclitaxel, ethanol and polyethoxylated castor oil. An amount of each formulation, sufficient to deliver 48 mg paclitaxel/kg rat, was administered via oral gavage directly to the stomach of the rats. Blood samples were drawn at specified time points (0.5, 2, 4, 8, 24, 48, and 72 hr) into heparinized tubes and centrifuged. The plasma was removed and prepared for paclitaxel content High Performance Liquid Chromatography (HPLC) analysis by liquid-liquid extraction with diethyl ether. The ether was evaporated and samples were reconstituted in a HPLC mobile phase of Acetonitrile:Water (70:30) and injected directly into the HPLC machine. Plasma paclitaxel concentrations were plotted at the respective time points of sampling.

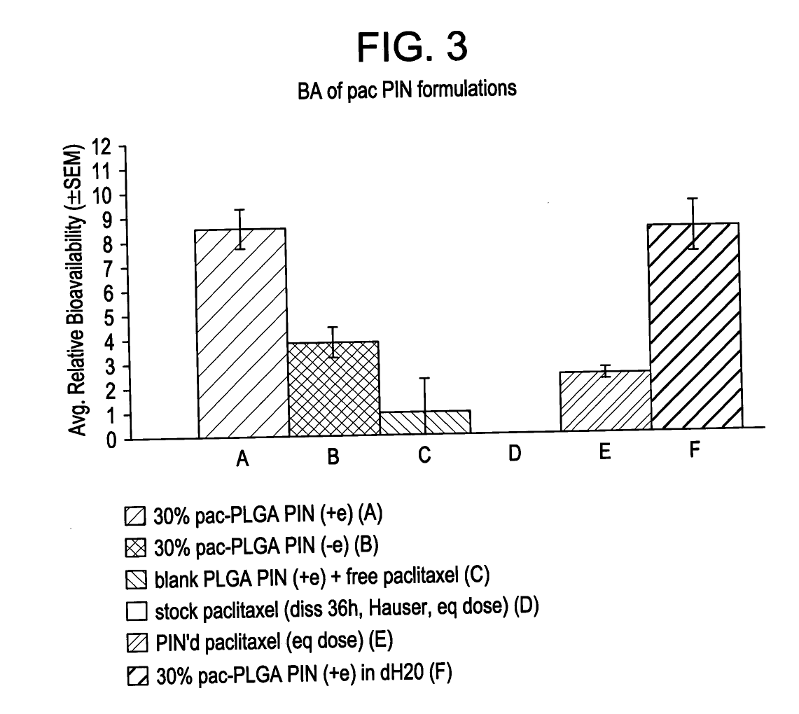

Since the low dose (<10 mg/kg) intravenous (IV) pharmacokinetics of paclitaxel are not linear, the bioavailability of the orally administered drug is calculated relative to the IV dose that yields the same plasma area under the curve (AUC) value as observed for a given oral administration. To accomplish this, IV pharmacokinetic studies were performed at several doses, the resultant AUC's were determined, and an equation-describing the dose/AUC relationship was fit to the data. This allows the calculation of the IV dose corresponding to the observed oral AUC. The fractional bioavailability (BA) of the oral dose is the ratio of the oral formulation's corresponding IV dose to the actual oral dose (IV dose/Oral dose). The results of this bioavailability study are shown in FIG. 3.

The Five Formulations and their Corresponding Relative Bioavailabilities

Paclitaxel microspheres were made essentially according to Example 2. In each case, the same amount (48 mg/kg) of paclitaxel was administered. In the leftmost column (a), the microspheres were taken up in isotonic phosphate buffered saline (PBS) containing 0.5% sodium lauryl sulfate (SLS) and 0.1% PVP (polyvinylpyrrolidone; MW 40,000 D). The relative bioavailability (BA) was 9.6%.

In the second column (b), the microspheres were made as described by Example 10, with a variation. No PVP was present in the paclitaxel solution. Instead, a separate solution containing PVP dissolved in dichloromethane (1% w/v) was precipitated in pentane. Then the PVP in pentane was immediately added to the drug particle suspension, at a mixing ratio designed to provide 1 mg of PVP (as a 1% solution diluted with 100 vol. of pentane) for 11.25 mg of paclitaxel (as a 3% solution diluted with 100 vol. of pentane), i.e., about 1 vol. of PVP for each 3.75 vol. of paclitaxel. (This gives the same ratio of PVP to paclitaxel as in Example 9.) After mixing the particulate paclitaxel and PVP solutions, the nanoparticles were recovered by filtration. The samples were taken up in 0.5% SLS in PBS, without additional PVP. When fed to rats, the observed BA was 10.2%.

In the center column (c), paclitaxel nanospheres were made as described by Example 2 and were taken up in 0.5% SLS in PBS. No additional surfactant was added. The observed BA was only 2.4%. This low BA may be due to incomplete redispersion of the nanoparticles in the absence of a suitable surfactant.

In the fourth (blank) column (d), the rats were fed stock (as purchased) paclitaxel that had been soaked in 0.5% SLS in PBS for 36 hours. The observed BA was 0%—no paclitaxel was found in the serum.

In the rightmost column (e), stock paclitaxel was taken up in 0.5% SLS in PBS and immediately administered. A BA of 0.7% was observed.

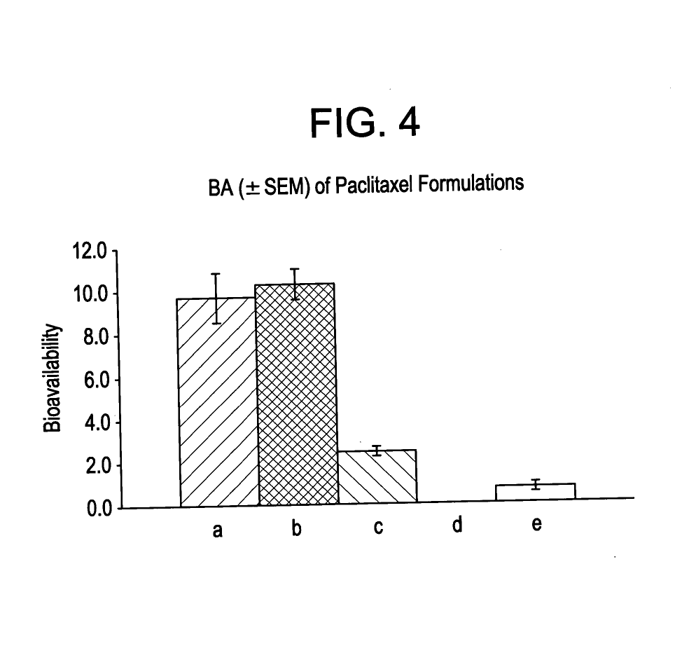

These results are graphically depicted in FIG. 4.

### Example 18

### Effect of Time of Sonication, Stability (Time After Sonication) and Solvent:non-solvent Ratio on Particle Size

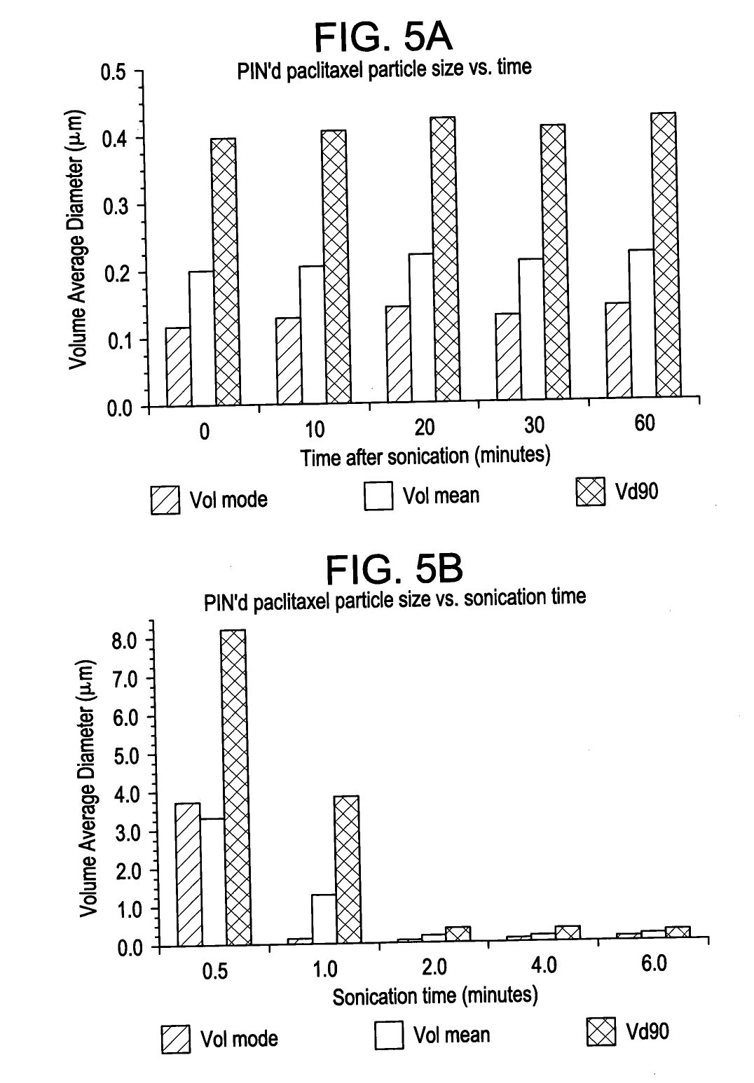

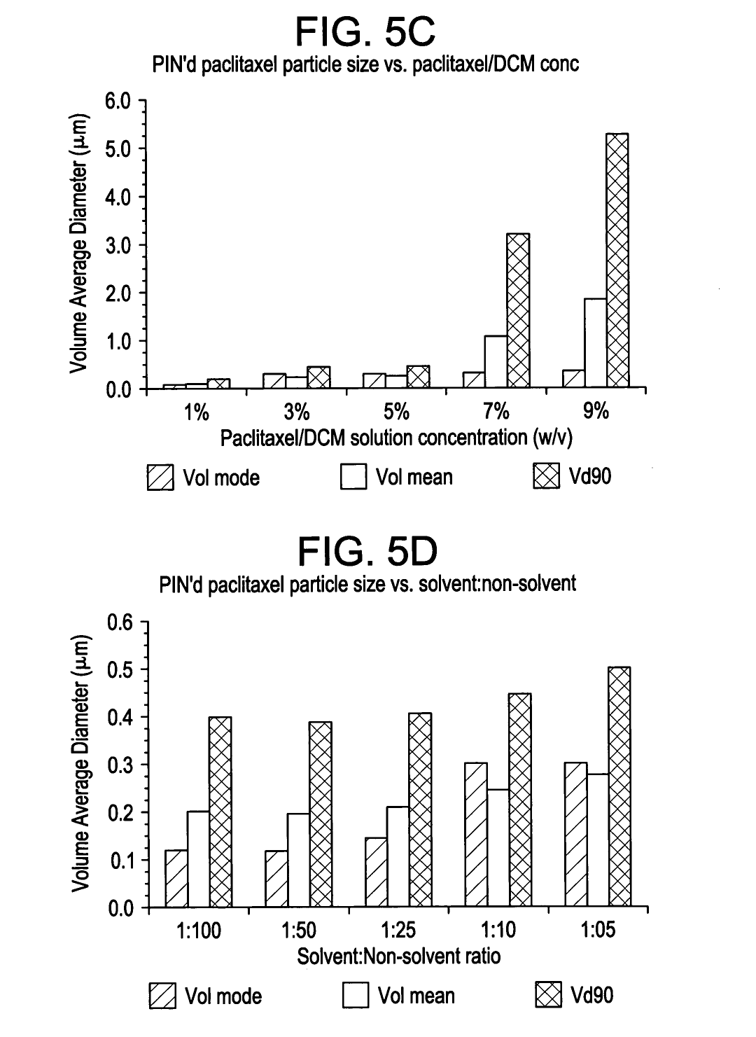

The effect of time after particle re-suspension following a single 3-minute bath sonication on size of paclitaxel particles prepared by PIN. All particles were prepared from a solution of 3% paclitaxel in methylene chloride with 100 volumes pentane at room temperature. Particles were recovered by filtration and vacuum dried. The particles were resuspended in 1.0% (w/v) PVP/0.5%(w/v) sodium laurel sulfate (“SLS”)/phosphate buffered saline (“PBS”). The results are shown in FIG. 5 a. This experiment shows the stability of particle size (absence of re-aggregation) after 3 minutes of bath sonication. (Note that the size scale on the left vertical axis is changed.) The results indicate that the particles are stable following sonication, at least up to 60 minutes.

The effect of bath sonication time on size of paclitaxel particles prepared by PIN. Samples were re-suspended and bath-sonicated for a specific of time and then immediately measured. Particle sizing re-suspension medium was 1.0%(w/v) PVP/0.5%(w/v) SLS/PBS. The results are shown in FIG. 5b. The results demonstrate that under the conditions used, sonication times of greater than one minute are required to yield nanometer diameters. Three minutes was selected as the standard sonication time. Note that by two minutes, under these conditions, the effective particle size was reduced to submicron values clustering around 0.1 micron.

Using the conditions established by these experiments, the effects of drug concentration and of dilution ratio in non-solvent were explored, using paxclitaxel as a model drug. The control condition in all experiments was 3% drug concentration, 1:100 dilution ratio in precipitation, and 3 min., of bath sonication after resuspension.

Drug concentration affects particle size. The concentration of paclitaxel in the methylene chloride was varied. The precipitation ratio was 1 vol. methylene chloride to 100 vol. pentane. Particles were collected, dried, resuspended, and sonicated for three minutes. The effect of paclitaxel solution concentration (w/v) in dichloromethane (DCM) (prior to PIN fabrication) on size of paclitaxel particles prepared by PIN was measured. Samples were re-suspended and then measured immediately following a single 3-minute bath sonication. The results are shown in FIG. 5c, and indicate that the lower concentrations of DCM (less than 7%) yield smaller diameter particle sizes, in the nanometer size range.

Effective particle size increased as drug loading increased. The increase was modest between 1% and 5% drug. The size distributions show a gradual shift from 100 nm particles to particles of about 500 nm diameter. At 7% and 9%, significant aggregation occurred, as judged by the appearance of 5 to 10 micron particles in the size distribution plot.

The effect of the solvent:non-solvent ratio (during the PIN process) on the size of paclitaxel particles prepared by PIN was determined. The solvent was dichloromethane and the non-solvent was pentane. Samples were re-suspended and then measured immediately following a single 3-minute 3-minute bath sonication. Particle sizing re-suspension medium was 1.0%(w/v) PVP/0.5%(w/v) SLS/PBS. FIG. 5d shows the results for ratios of solvent dichloromethane:non-solvent pentane of 1:100 to 1:05. All ratios yielded nanoparticles. Dilution ratios of 1:100 (standard), 1:50, 1:25, 1:10 and 1:5 are illustrated. The 1:100 and 1:50 dilutions are essentially identical; the 1:25 dilution shows some increase in 0.3-0.5 micron range particles; and the particle size mode shifts to the 0.5 micron range at 1:10 and 1:5. The size distribution plot shows that this increase was not accompanied by significant expansion of particle size into the multi-micron size range. The effect of additional sonication was not explored. A dilution ratio of 1:1 was also tested. The drug precipitated as a macroscopic precipitate, with little evidence of fine particle creation; in one experiment, the precipitate spontaneously redissolved. implying that the 1:1 ratio is very close to the limiting concentration of pentane in dichloromethane in which paclitaxel is soluble. Temperature can also be a variable in solubility.

This data set shows that to obtain final particles in the 100 nm range, with paclitaxel in dichloromethane precipitated with pentane, a concentration of 1% at a dilution ratio of 50 is approximately optimal. For 500 nm particles, stable against mild sonication, a 5% concentration of drug and a dilution ratio of 1:5 is approximately optimal, in terms of minimization of solvent use.

In other experiments, 3% of the drug carbamazepine in dichloromethane was precipitated in 100 vol. of pentane. By electron microscopy, the particles were predominantly needles, about 1 micron in diameter by 50 microns in length. A few 1 micron spherical particles were also observed. A similar experiment using the drug itraconazole produced generally spherical particles in the range of about 0.1 to 0.5 microns diameter, by electron microscopy.

These experiments demonstrate that particular conditions are required for the reliable formation of nanoparticles, and that the formation of nanoparticles is not a necessary result of dissolving a drug in a solvent and precipitating it into a nonsolvent. It also demonstrates that once in possession of information of a general range in which nanoparticles can be obtained, optimization of conditions for a particular drug requires only a reasonable amount of experimentation.

### Example 19

### Effect on Tumor Growth of Paclitaxel Nanoparticles Administered Orally to Mice

Paclitaxel nanoparticles prepared as described above (no bioadhesive or surfactant) were orally administered b.i.d.×5 to female nude mice innoculated with breast tumor cells. The nanoparticles were resuspended in 0.1%PVP/0.5% SLS/PBS at 11.25 mg/ml and administered by oral gavage to provide theoretical dosages of 24, 48 and 72 mg/kg. The control was Taxol in cremaphor administered by intravenous injection (30 mg/kg).

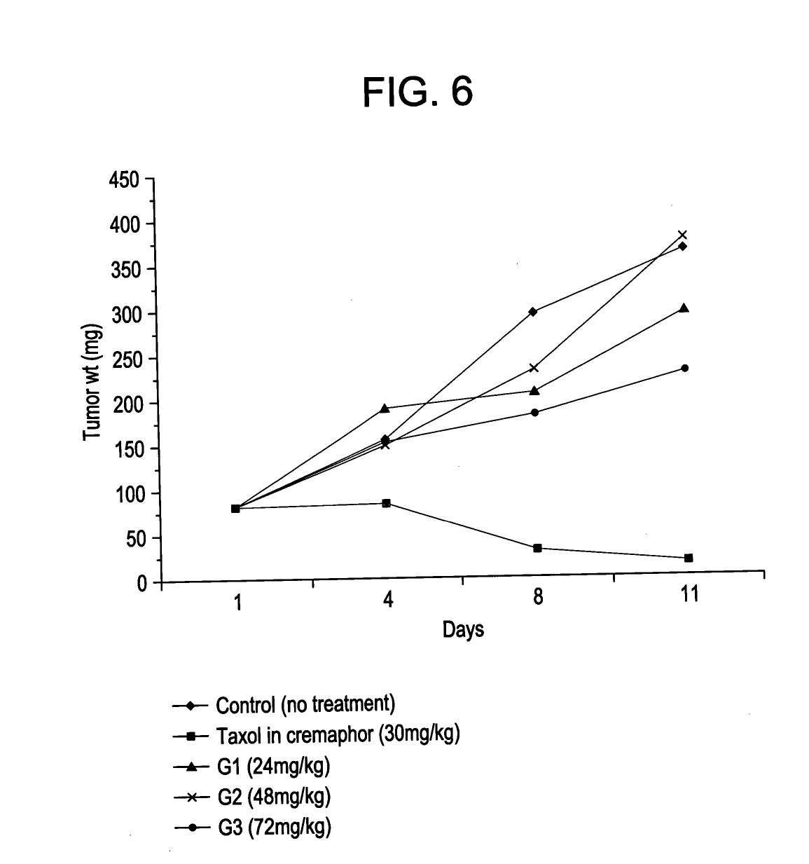

The results are shown in FIG. 6. The results indicate that the Taxol nanoparticles had an approximate bioavailability of 17%, relative to the Taxol in cremaphor, and caused some reduction in tumor growth, with approximately 50% reduction in tumor volume at the 72 mg/kg dosage. Variables which can be used to increase efficacy include higher drug loading, incorporation of bioadhesives, and optimization of particle size (less than 500 nm) to increase uptake.

Those skilled in the art will recognize, or be able to ascertain using no more than routine experimentation, many equivalents to the specific embodiments of the invention described herein. Such equivalents are intended to be encompassed by the following claims.

We claim:

1. A method for preparing nanoparticles of a therapeutic, prophylactic or diagnostic agent, comprising
dissolving the agent in a solvent to form a first solution,
providing a non-solvent for the agent which is miscible with the solvent, and
mixing the first solution with the non-solvent to form nanoparticles of the therapeutic, prophylactic or diagnostic agent, wherein the nanoparticles form a population of which at least 95% has a diameter of less than one micron.

2. The method of claim 1, further comprising adding a surfactant or excipient.

3. The method of claim 2, wherein the surfactant or excipient is added to the solvent.

4. The method of claim 2, wherein the surfactant or excipient is added to the non-solvent.

5. The method of claim 2, wherein the surfactant or excipient is added to the nanoparticles after their formation.

6. The method of claim 1, wherein the agent is selected from the group consisting of small-molecule drugs, proteins, lipids, polysaccharides, proteoglycans, and polynucleotides.

7. The method of claim 1, wherein the agent is soluble in water to less than about 0.1% w/v at room temperature.

8. The method of claim 1, wherein the agent is sufficiently hydrophobic to be insoluble in water.

9. The method of claim 1, further comprising collecting the nanoparticles by centrifugation, filtration, lyophilization, or spray drying.

10. The method of claim 1, wherein less than about 1% of the nanoparticles have a diameter of greater than about 1 micron.

11. A population comprising at least 95% nanoparticles of a therapeutic, diagnostic or prophylactic agent having a diameter of less than one micron.

12. The population of claim 11, wherein the agent is selected from the group consisting of small-molecule drugs, proteins, lipids, polysaccharides, proteoglycans, and polynucleotides.

13. The population of claim 11, wherein the agent is soluble in water to less than about 0. 1% w/v at room temperature.

14. The population of claim 11, wherein the agent is sufficiently hydrophobic to be insoluble in water.

15. The population of claim 11 wherein at least 99% of the nanoparticles have a diameter of less than one micron.

17. The formulation of claim 11 further comprising bioadhesive enhancing agents.

18. The formulation of claim 11 further comprising a dispersant.

19. The formulation of claim 11 further comprising a polymer.

20. The formulation of claim 11 comprising a polymer encapsulated agent having bioadhesive agent bound thereto or dispersed therein.

21. The formulation of claim 17 wherein the bioadhesive agent is selected from the group consisting of bioadhesive metal compounds and bioadhesive organic molecules.

22. The formulation of claim 11, wherein the nanoparticles are formed by a method comprising
dissolving the bioactive agent in a solvent to form a first solution;
providing a non-solvent for the bioactive agent, wherein the non-solvent is miscible with the solvent; and
mixing the first solution with the non-solvent to form nanoparticles.

23. A nano or microparticulate formulation for oral administration of a taxane providing a bioavailability of at least 5% of the bioavailability of the taxane when administered intravenously.

24. The formulation of claim 23 wherein the taxane is paclitaxel.

25. The formulation of claim 23 wherein the taxane is docetaxel.

26. The formulation of claim 23 wherein 90%, by volume or number, of the nanoparticles and microparticles have a diameter of less than five microns.

27. The formulation of claim 23 wherein 90%, by volume or number, of the nanoparticles and microparticles have a diameter of less than one micron.

28. The formulation of claim 23 wherein the taxane is present in a drug loading of up to 70% by weight.

29. The formulation of claim 23 wherein the taxane is present in a drug loading of between approximately 30 and 70% by weight.

30. The formulation of claim 23 further comprising a surfactant or excipient.

31. A method for treating a patient comprising administering the nanoparticle formulation of claim 11 or 23 to a patient.

32. The method of claim 31, wherein the formulation is selected from the group consisting of oral formulations, aerosols, topical formulations, parenteral formulations, and implantable compositions.

33. The method of claim 31 wherein the formulation is administered orally.

34. The method of claim 31 wherein the formulation is administered to the pulmonary system.
